(19) United States
(12) Patent Application Publication
(10) Pub. No.: US 2024/0342364 A1
(43) Pub. Date: Oct. 17, 2024

(54) Drug Delivery Device with Cannula Having Bioactive Agent

(71) Applicant: Becton, Dickinson and Company, Franklin Lakes, NJ (US)

(72) Inventors: Steve Beguin, Nenzlingen (CH); Stuart Plascott, Foxrock (IE); Ronald J. Pettis, Cary, NC (US); Natasha G. Bolick, Raleigh, NC (US); John Adams, Killester (IE)

(21) Appl. No.: 18/682,965

(22) PCT Filed: Aug. 12, 2022
(86) PCT No.: PCT/US22/40186

§ 371 Date: Feb. 12, 2024

Related U.S. Application Data
(60) Provisional application No. 63/232,732, filed on Aug. 13, 2021.

(51) Int. Cl. A61M 5/142; A61K 45/06; A61M 5/168

(52) U.S. Cl. CPC A61M 5/14248; A61K 45/06; A61M 5/16831; A61M 2005/14252; A61M 2005/16863; A61M 2205/0238; A61M 2205/10; A61M 2205/3379; A61M 2205/50; A61M 2205/8206

(57) ABSTRACT

A drug delivery device includes a reservoir configured to receive a fluid, a cannula in fluid communication with the reservoir, with the cannula configured to be inserted into subcutaneous tissue or muscle tissue of a patient, and a pump configured to deliver a fluid from the reservoir to the cannula. The cannula includes a bioactive agent configured to cause a tissue response to decrease a pressure required to deliver fluid from the reservoir. The tissue response can include vasodilation, vasoconstriction, increased tissue permeability, increased flow of interstitial fluid or enzymatic deterioration of extracellular matrix.

### CROSS-REFERENCE TO RELATED APPLICATION

The present application claims priority to U.S. Provisional Application Ser. No. 63/232,732, filed Aug. 13, 2021, entitled “Drug Delivery Device with Cannula Having Bioactive Agent”, the entire disclosure of which is hereby incorporated by reference in its' entirety.

### BACKGROUND OF THE INVENTION

### Field of the Invention

The present disclosure relates to a drug delivery device with a cannula having a bioactive agent.

### Description of Related Art

Wearable medical devices, such as automatic injectors, have the benefit of providing therapy to the patient at a location remote from a clinical facility and/or while being worn discretely under the patient's clothing. The wearable medical device can be applied to the patient's skin and configured to automatically, or at user's command, deliver a dose of a pharmaceutical composition within a predetermined time period after applying the wearable medical device to the patient's skin, such as after a 27 hour delay or immediately upon actuation of the device. After the device delivers the pharmaceutical composition to the patient, the patient may subsequently remove and dispose of the device.

In certain circumstances, due to the medium in which the liquid is being injected, the flow of fluid leaving the device may be impaired, which can lead to increased pressure in the fluid line of the device. When the pressure rises above a certain threshold, the integrity of the fluid path may be compromised causing a leak within the device and a failure to deliver the full dose of medicament. A fluid leak within the device may also cause damage to the device and subsequent system failures as well as potential contamination concerns due to contact between the fluid and the device.

Human subcutaneous tissue is composed of various cell types, extracellular matrix (ECM) constituents, microstructures, and macroscopic arrangement of cells and ECM. Those elements contribute to the mechanical properties of the tissue. The tissue may also include lymphatic system and blood vessels, and has intrinsic fluid absorption and retention properties. These characteristics vary among individuals, location within the body, and over time may cause variable degrees of resistance to the infusion of fluids at the site of injection. When the resistance of the tissue is too high or the absorption rate is too low for a given delivery flow rate from the device, the pressure may build up and reach values above the threshold where the fluid line and other components may be compromised.

### SUMMARY OF THE INVENTION

In one aspect or embodiment, a drug delivery device includes a reservoir configured to receive a fluid, a cannula in fluid communication with the reservoir, the cannula configured to be inserted into subcutaneous tissue or muscle tissue of a patient, and a pump configured to deliver a fluid from the reservoir to the cannula, where the cannula includes a bioactive agent configured to cause a tissue response to decrease a pressure required to deliver fluid from the reservoir.

The cannula may be at least one of a catheter and a needle. In certain embodiments, the cannula may comprise multiple cannulae. The tissue response may be at least one of vasodilatation, vasoconstriction, increased tissue permeability, increased flow of interstitial fluid, and enzymatic deterioration of extracellular matrix. The bioactive agent may be at least one of a nitrovasodilator, a cytokine, an enzyme, a histamine, and a chemical irritant. The nitrovasodilator may be at least one of nitrate, niacinamide, and nitroprusside. The cytokine may include tumor necrosis factor alpha. The chemical irritant may be at least one of capsaicin and camphor. The enzyme may be hyaluronidase.

The bioactive agent may be configured to immediately initiate a tissue response upon insertion of the cannula into subcutaneous tissue or muscle tissue of a patient.

The bioactive agent may be configured to prevent encapsulation of the cannula and prevent bio formed tissue encapsulation which may alter the permeability of the tissue or impede the fluid path or fluid egress. The bioactive agent may include at least one of a TGF inhibitor (e.g., poly-arginine), VEGF inhibitor, or substance configured to prevent collagen excretion from cells.

The bioactive agent may be positioned on an inside surface or outside surface of the cannula. The bioactive agent may include a material forming the cannula or a secondary metabolite arising from the degradation of a material forming the cannula. The bioactive agent may be embedded within the cannula, with the bioactive agent configured to be released at a controlled rate upon insertion of the cannula into subcutaneous tissue or muscle tissue of a patient.

The cannula may include a coating, with the bioactive agent carried in the coating. The coating may be a frangible coating. The coating may be a cross-linked siliconized coating.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a drug delivery device according to one aspect or embodiment of the present application.

FIG. 2 is a perspective view of the drug delivery device of FIG. 1, with a top cover removed.

FIG. 3 is a schematic of the drug delivery device of FIG. 1.

FIG. 4 is a cross-sectional view of the drug delivery device of FIG. 1.

FIG. 5 is a perspective view of a drug delivery device according to a further aspect or embodiment of the present application.

FIG. 6 is a cross-sectional view taken along line 6-6 shown in FIG. 5.

FIG. 7 is a cross-sectional view taken along line 7-7 shown in FIG. 5.

FIG. 8A is a schematic view of subcutaneous tissue of a patient, showing a first state prior to introduction of a bioactive agent.

FIG. 8B is a schematic view of subcutaneous tissue of a patient, showing a second state after introduction of a bioactive agent.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

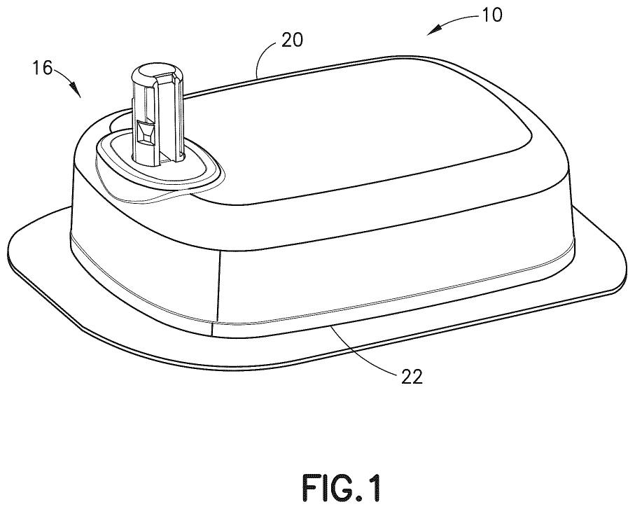

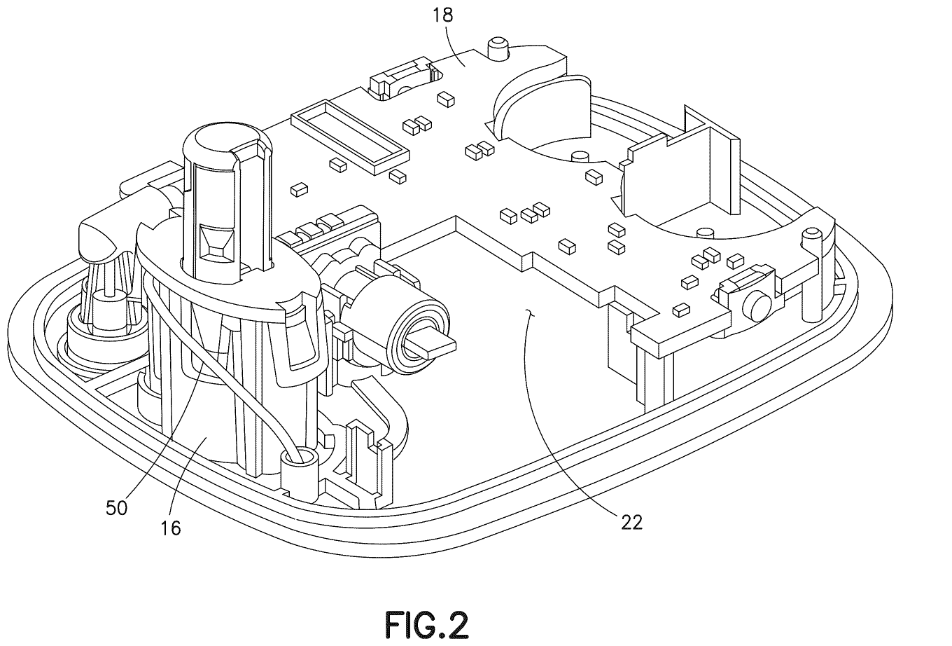

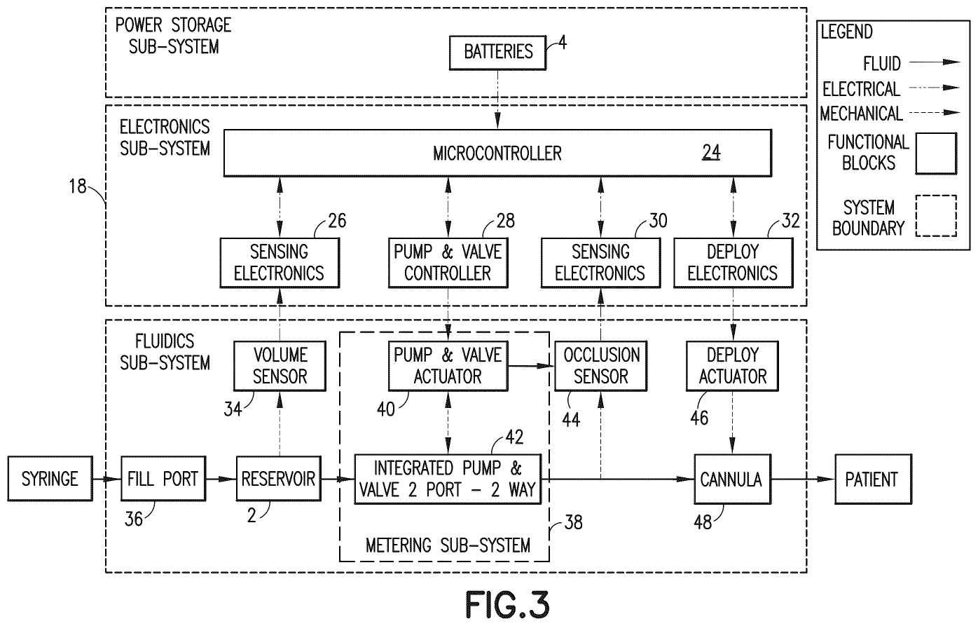

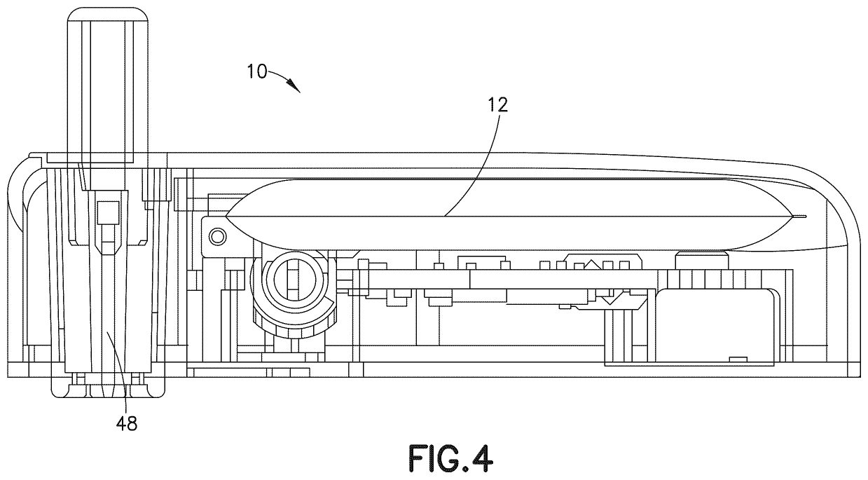

Referring to FIGS. 1-4, a drug delivery device 10 includes a reservoir 12, a power source 14, an insertion mechanism 16, control electronics 18, a cover 20, and a base 22. In one aspect or embodiment, the drug delivery device 10 is a wearable automatic injector, such as an insulin or bone marrow stimulant delivery device. The drug delivery device 10 may be mounted onto the skin of a patient and triggered to inject a pharmaceutical composition from the reservoir 12 into the patient. The drug delivery device 10 may be pre-filled with the pharmaceutical composition, or it may be filled with the pharmaceutical composition by the patient or medical professional prior to use.

The drug delivery device 10 is configured to deliver a dose of a pharmaceutical composition, e.g., any desired medicament, into the patient's body by a subcutaneous injection at a slow, controlled injection rate. Exemplary time durations for the delivery achieved by the drug delivery device 10 may range from about 5 minutes to about 60 minutes, but are not limited to this exemplary range. Exemplary volumes of the pharmaceutical composition delivered by the drug delivery device 10 may range from about 0.1 milliliters to about 10 milliliters, but are not limited to this exemplary range. The volume of the pharmaceutical composition delivered to the patient may be adjusted.

Referring again to FIGS. 1-4, in one aspect or embodiment, the power source 14 is a DC power source including one or more batteries. The control electronics 18 include a microcontroller 24, sensing electronics 26, a pump and valve controller 28, sensing electronics 30, and deployments electronics 32, which control the actuation of the drug delivery device 10. The drug delivery device 10 includes a fluidics sub-system that includes the reservoir 12, a volume sensor 34 for the reservoir 12, a reservoir fill port 36, and a metering sub-system 38 including a pump and valve actuator 40 and a pump and valve mechanism 42. The fluidic sub-system may further include an occlusion sensor 44, a deploy actuator 46, a cannula 48 for insertion into a patient's skin, and a fluid line 50 in fluid communication with the reservoir 12 and the cannula 48. In one aspect or embodiment, the insertion mechanism 16 is configured to move the cannula 48 from a retracted position positioned entirely within the device 10 to an extended position where the cannula 48 extends outside of the device 10. In one aspect or embodiment, the cannula 48 is a catheter, which may be a soft, flexible catheter, a rigid catheter, or a combination thereof. The drug delivery device 10 may operate in the same manner as discussed in U.S. Pat. No. 10,449,292 to Pizzochero et al.

In further aspects or embodiments, the drug delivery device 10 is a pen injector, auto-injector, or a syringe.

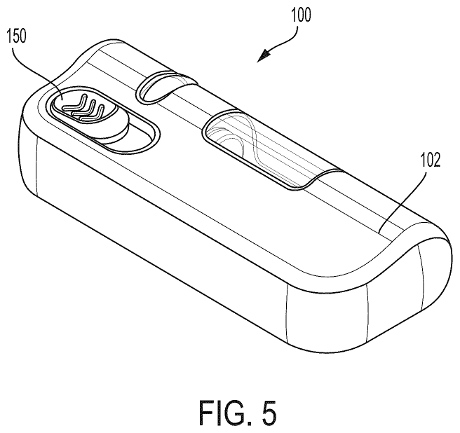

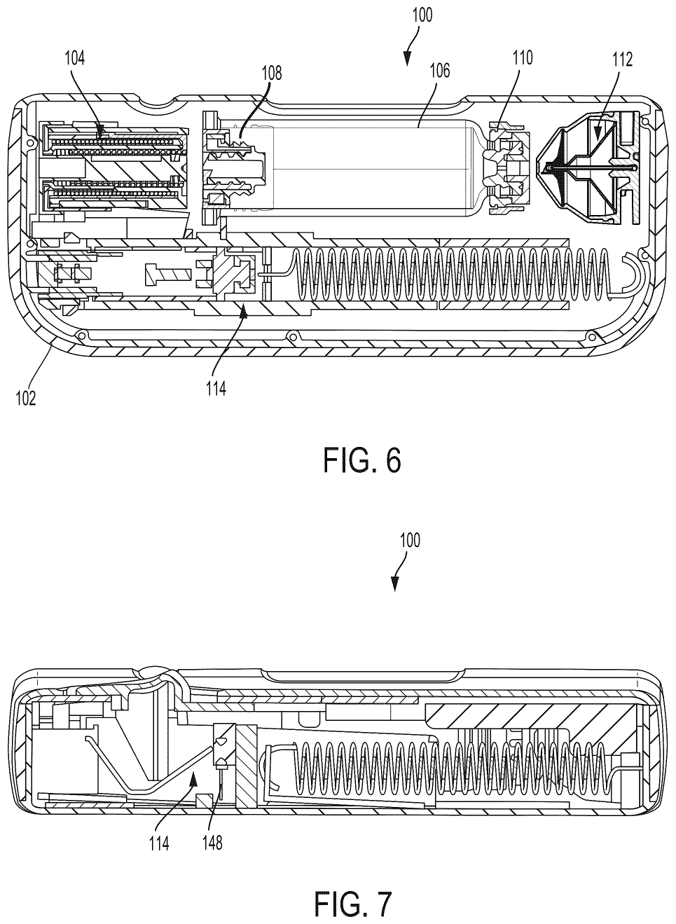

Referring to FIGS. 5-7, in a further aspect or embodiment, a drug delivery device 100 includes a housing 102, a pump assembly 104, a reservoir 106 having a stopper 108 and a closure 110, a valve assembly 112, and a needle actuation assembly 114 including a cannula 148. Although one cannula 148 is shown, the cannula 148 may include one or more cannulas. Actuation of the drug delivery device 100 via a button 150 causes the needle actuation assembly 114 to move the cannula 148 from a retracted position to an extended position while simultaneously causing the pump assembly 104 to move the reservoir 106 into engagement with the valve assembly 112, which, in turn, punctures the closure 110 of the reservoir 106. Upon puncturing of the closure 110 of the reservoir 106, the pump assembly 104 is in fluid communication with the cannula 148 via flow tube (not shown), which allows the pump assembly 104 to move the stopper 108 within the reservoir 106 to deliver fluid from the reservoir 106 to the cannula 148. In use, the drug delivery device 100 is worn or placed on a skin surface of a patient with the button 150 actuating the device 100 to deliver a dose of medication within the reservoir 106 to the patient via the cannula 148, which is inserted into the patient. In one aspect or embodiment, the cannula 148 is a needle. After completion of delivery of the dose of medication, the cannula 148 is automatically retracted within the housing 102 of the drug delivery device 100.

In one aspect or embodiment, the pump assembly 104 is driven by one or more springs, although other suitable power sources or arrangements may be utilized. In one aspect or embodiment, the reservoir 106 is a container having a cylindrical barrel. It is further contemplated herein that the cannula 148 may be supported by a hub, which may be in communication with a remote fluid source that is not housed within the same structure as the cannula. In certain configurations, the hub and supported cannula may be connected to the remote fluid source by a length of tubing. For example, the cannula may be supported by a hub in communication with a remote infusion pump, IV bag, or the like.

In one aspect or embodiment of present application, the cannulas 48,148 of the drug delivery devices 10,100 include a bioactive agent configured to cause a tissue response to decrease a pressure required to deliver fluid from the reservoir 12,108. The cannulas 48,148, as described above, are configured to be inserted into subcutaneous tissue. In a further aspect or embodiment, the cannulas 48,148 are configured to be inserted into muscle tissue of a patient. The bioactive agent may be configured to elicit a tissue response to render the tissue more porous, more vascularized, more perfused, and/or weaker in order to decrease the pressure required to infiltrate the tissue where the drug is being injected by the drug delivery devices 10,100.

In one aspect or embodiment, the bioactive agent is configured to reduce a peak pressure required by the drug delivery devices 10,100 to deliver fluid from the reservoirs 12,108. In one aspect or embodiment, the peak pressure is reduced below 50 psi. In one aspect or embodiment, the peak pressure is reduced below 40 psi. In one aspect or embodiment, the peak pressure is reduced below 10 psi.

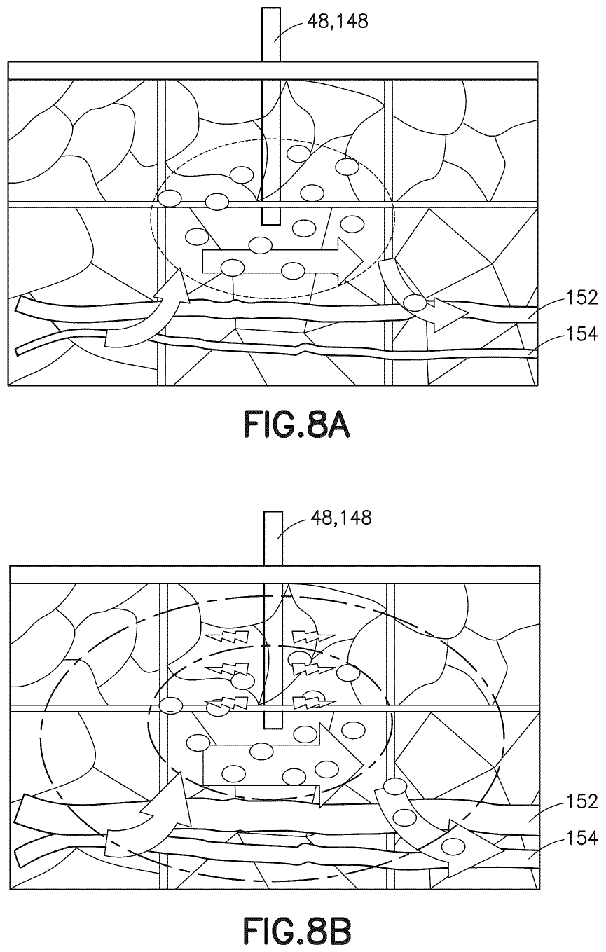

In one aspect or embodiment, the tissue response caused by the bioactive agent includes at least one of vasodilatation, vasoconstriction, increased tissue permeability, increased flow of interstitial fluid, increased reuptake of interstitial fluid by lymphatic system, and enzymatic deterioration of extracellular matrix. Increasing the permeability may help diminish local accumulation of depot and limit surface tissue response (e.g., wheal, bleb, swelling, induration, erythema, or bruising) formation or accelerate their resolution. A first state of the subcutaneous tissue of the patient prior to the introduction of the bioactive agent is shown in FIG. 8A. In a second state of the subcutaneous tissue of the patient after introduction of the bioactive agent, which is shown in FIG. 8B, the bioactive agent causes a tissue response to improve the drug absorption rate. For example, the bioactive agent may provide a vasodilated lymph capillary 152 and/or a vasodilated blood capillary 154, as shown in FIG. 8B.

In one aspect or embodiment, the bioactive agent includes at least one of a vasodilator, nitrovasodilator, a cytokine, an enzyme, a histamine, and a chemical irritant. The nitrovasodilator may include at least one of nitrate, niacinamide, and nitroprusside. The cytokine may be tumor necrosis factor alpha (TNF-α). The chemical irritant may be at least one of capsaicin and camphor. The chemical irritant may be configured to cause vasodilation via simulated heat while minimizing the sensation of pain for the patient. The enzyme may be hyaluronidase, although other suitable enzymes to weaken mechanical properties of the tissue may be utilized. The bioactive agent may have anticoagulating, pro-inflammatory, or other properties inducing chemotaxis of either immune or non-immune cells. The bioactive agent may interact with, degrade, or remodel the extracellular matrix, e.g., matrix metalloproteinase, to make it more porous or weaker in order to render it more permeable to the drug being injected. The bioactive agent may act as retaining or capturing water from the surrounding tissue. The bioactive agent may induce local inflammation. The bioactive agent may induce vasodilation of blood capillaries and/or lymphatic capillaries to selectively induce either vasodilation or vasoconstriction to only blood capillaries or only lymphatic capillaries, and any combination thereof.

In one aspect or embodiment, the bioactive agent is hyaluronic acid and configured to alter the water content of the target tissue.

In one aspect or embodiment, the bioactive agent is configured to immediately initiate a tissue response upon insertion of the cannulas 48,148 into subcutaneous tissue or muscle tissue of a patient. In another aspect or embodiment, the bioactive agent is configured to be released over a predetermined period of time. The predetermined period of time may be 1 hour, 5 hours, 10 hours, 15 hours, 24 hours, 25 hours, 26 hours, 27 hours, or longer. In one aspect or embodiment, the predetermined period of time is 3-7 days.

In one aspect or embodiment, the bioactive agent is positioned on an inside surface or outside surface of the cannulas 48,148. In a further aspect or embodiment, the bioactive agent is provided as a material forming the cannulas 48,148 or a secondary metabolite arising from the degradation of a material forming the cannulas 48,148. A material of the cannulas 48,148 may be a polymer, including polyactic acid or poly lactic-co-glycolic acid polymers.

In one aspect or embodiment, the bioactive agent is embedded within the cannulas 48,148, with the bioactive agent configured to be released at a controlled rate upon insertion of the cannulas 48,148 into subcutaneous tissue or muscle tissue of a patient. The bioactive agent may be embedded in a polymer to allow release at a controlled rate. In one aspect or embodiment, the bioactive agent is embedded in a material, such as a polymer, of the cannulas 48,148 and the cannulas 48,148 are also at least partially coated with the bioactive agent, which provides an immediate tissue response as well as a controlled release of the bioactive agent. In one aspect or embodiment, the bioactive agent is an enzyme, such as hyaluronidase, embedded within a material of the cannulas 48,148, with the enzyme configured to disrupt the tissue matrix and the release of the enzyme configured to correspond with the time of injection.

In one aspect or embodiment, the cannulas 48,148 include a coating, with the bioactive agent carried in the coating. The coating may be a frangible coating. The frangible coating may be a mono-, di- and triglyceride esters of fatty acids (C10-C18) with and without surfactants, to make the frangible coatings. In a further aspect or embodiment, the coating is a cross-linked siliconized coating. The bioactive agent may be dispersed in the siliconized coating and cross-linked via heat and/or radiation without degrading the bioactive agent to provide both lubrication for insertion of the cannulas 48,148 and also provide the tissue response.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A drug delivery device comprising:
a reservoir configured to receive a fluid;
a cannula in fluid communication with the reservoir, the cannula configured to be inserted into subcutaneous tissue or muscle tissue of a patient; and
a pump configured to deliver a fluid from the reservoir to the cannula,
wherein the cannula comprises a bioactive agent configured to cause a tissue response to decrease a pressure required to deliver fluid from the reservoir.

2. The drug delivery device of claim 1, wherein the cannula comprises at least one of a catheter and a needle.

3. The drug delivery device of claim 1, wherein the tissue response comprises at least one of vasodilation, vasoconstriction, increased tissue permeability, increased flow of interstitial fluid, and enzymatic deterioration of extracellular matrix.

4. The drug delivery device of claim 1, wherein the bioactive agent comprises at least one of a vasodilator, nitrovasodilator, a cytokine, an enzyme, a histamine, and a chemical irritant.

5. The drug delivery device of claim 4, wherein the nitrovasodilator comprises at least one of nitrate, niacinamide, and nitroprusside.

6. The drug delivery device of claim 4, wherein the cytokine comprises tumor necrosis factor alpha.

7. The drug delivery device of claim 4, wherein the chemical irritant comprises at least one of capsaicin and camphor.

8. The drug delivery device of claim 4, wherein the enzyme comprises hyaluronidase.

9. The drug delivery device of claim 1, wherein the bioactive agent is configured to immediately initiate a tissue response upon insertion of the cannula into subcutaneous tissue or muscle tissue of a patient.

10. The drug delivery device of claim 1, wherein the bioactive agent is positioned on an inside surface or outside surface of the cannula.

11. The drug delivery device of claim 1, wherein the bioactive agent comprises a material forming the cannula or a secondary metabolite arising from the degradation of a material forming the cannula.

12. The drug delivery device of claim 1, wherein the bioactive agent is embedded within the cannula, the bioactive agent configured to be released at a controlled rate upon insertion of the cannula into subcutaneous tissue or muscle tissue of a patient.

13. The drug delivery device of claim 1, wherein the cannula comprises a coating, the bioactive agent carried in the coating.

14. The drug delivery device of claim 13, wherein the coating comprises a frangible coating.

15. The drug delivery device of claim 13, wherein the coating comprises a cross-linked siliconized coating.

16. The drug delivery device of claim 1, wherein the bioactive agent is configured to prevent encapsulation of the cannula and prevent bio formed tissue encapsulation.

17. The drug delivery device of claim 16, wherein the bioactive agent comprises at least one of a TGF inhibitor and a VEGF inhibitor.

18. A drug delivery device comprising:
at least one penetrating body configured to be inserted into the subcutaneous tissue or muscle of a patient;
wherein the at least one penetrating body comprises a bioactive agent configured to cause a tissue response to decrease a pressure required to deliver a fluid into the tissue or muscle of the patient.

19. The drug delivery device of claim 18, wherein the penetrating body comprises at least one cannulae.

20. The drug delivery device of claim 18, wherein the bioactive agent comprises at least one of a vasodilator, nitrovasodilator, a cytokine, an enzyme, a histamine, and a chemical irritant.

21.-22. (canceled)
